(19) United States
(12) Patent Application Publication
(10) Pub. No.: US 2004/0209364 A1
(43) Pub. Date: Oct. 21, 2004

(54) AAV vector packaging plasmid for producing wtaav particles or pseudotyped aav particles without helper viruses, by means of a single transfection

(76) Inventors: Dirk Grimm, Ludwigshafen (DE); Jurgen kleinschmitt, Bammental (DE)

Correspondence Address: INTELLECTUAL PROPERTY / TECHNOLOGY LAW, PO BOX 14329, RESEARCH TRIANGLE PARK, NC, 27709, US

(21) Appl. No.: 10/485,937

(22) PCT Filed: May 8, 2002
(86) PCT No.: PCT/DE02/01670

(30) Foreign Application Priority Data
Aug. 1, 2001 (DE) 101 37 283.3

(51) Int. Cl. C12N 7/01; C12N 15/861

(52) U.S. Cl. 435/456; 435/235.1; 435/457

(57) ABSTRACT

The present invention relates to AAV vector packaging plasmids for the helper virus-free preparation of (pseudotyped) AAV particles by means of single transfection. The AAV vector packaging plasmids for the (pseudotyped) AAV particles comprise the following DNA sequences: (a) a rep gene of AAV, (b) a cap gene of AAV, (c) AAV expression vector DNA sequences, and (d) all further helper virus DNA sequences necessary for forming AAV particles. The AAV vector packaging plasmids for the preparation of wtAAV particles are characterized in that they (a) comprise the complete AAV genome and (b) all of the helper virus DNA sequences necessary for forming AAV particles. The invention also relates to the use of these AAV vector packaging plasmids for preparing wtAAV particles or pseudotyped AAV particles in particular for gene therapy or tumor therapy.

The present invention relates to AAV vector packaging plasmids for the helper virus-free preparation of (pseudotyped) AAV particles by means of single transfection. The AAV vector packaging plasmids for the (pseudotyped) AAV particles comprise the following DNA sequences: (a) a rep gene of AAV, (b) a cap gene of AAV, (c) AAV expression vector DNA sequences, and (d) all further helper virus DNA sequences necessary for forming AAV particles. The AAV vector packaging plasmids for the preparation of wtAAV particles are characterized in that they comprise (a) the complete AAV genome and (b) all of the helper virus DNA sequences necessary for forming AAV particles. The present invention also relates to the use of these AAV vectors for the preparation of wtAAV particles or pseudotyped AAV particles, in particular for gene therapy or tumor therapy.

AAVs are single-stranded DNA viruses belonging to the parvovirus family. AAVs need helper viruses, in particular adenoviruses or herpes viruses, for their replication. In the absence of helper viruses, AAVs integrate into the host cell genome, in particular at a specific site of chromosome 19. The AAV genome is linear and has a length of about 4680 nucleotides. It comprises two reading frames coding for a structural and a non-structural gene. The structural gene is referred to as the cap gene. It is controlled by the P40 promoter and codes for three capsid proteins. The non-structural gene is referred to as rep gene and codes for the Rep proteins Rep 78, Rep 68, Rep 52 and Rep 40. The two former ones are expressed under the control of the P5 promoter, while the expression of Rep 52 and Rep 40 is controlled by the P19 promoter. The functions of the Rep proteins include inter alia the regulation of the replication and transcription of the AAV genome.

AAVs have been developed and tested intensively as possible vectors for human gene therapy for some time now. Among the six different AAV serotypes (AAV-1 to AAV-6) cloned and sequenced thus far, AAV-2 is the serotype which is characterized the best, and most of the vectors used for the time being are based on AAV-2. However, reports on the preparation and evaluation of the other five AAV serotypes have also been published in the past few years. It has turned out that ITRs (inverted terminal repeats) at either end of the AAV genome are the only cis elements necessary for the replication (i.e. excision of the viral DNA from the plasmid, replication and packaging of the intermediary DNA sequences) of AAV vectors (by means of the helper virus). Thus, it was obvious that in principle every DNA flanked by AAV-ITRs and comparable regarding its length with a wild-type viral genome can be packed into AAV capsids in the presence of the rep and cap gene products in trans and the helper virus functions. In most cases, a method was used for the production of these vectors, which has to use helper viruses, i.e. the cells are cotransfected with the AAV vector and helper plasmids and then infected with the helper adenovirus, which results in recombinant AAV vectors, yet contaminated with adenovirus. Another strategy is based on a triple transfection where in addition to avoiding a helper virus contamination a non-infectious adenoviral plasmid is used for the provision of helper functions.

In summary, there are currently three different approaches:

(a) cotransfection of AAV helper sequences providing the rep and cap genes of the respective AAV serotype and the corresponding vector plasmids of AAV-2, AAV-3, AAV-5 or AAV-6, (b) cotransfection of AAV-2 vector plasmids with AAV helper plasmids carrying the rep gene of AAV-2 and the cap gene of AAV-1, AAV-3 or AAV-5, and (c) cotransfection of AAV-2 vector plasmids with rep-cap genes of AAV-1, AAV-3, AAV-4 or AAV-6. The adenoviral helper functions are provided in all of the three approaches by infection with adenoviruses or by additional transfection of plasmids carrying the adenoviral genome. However, all of the current approaches have certain serious drawbacks. For example, (a) either different vector plasmids have to be used for the packaging into different AAV serotypes, (b) the vector production by triple infections is complicated and expensive, and (c) the double transfection and infection with adenoviruses raises the problem of adenovirus contamination.

The problem on which the present invention is based was thus to provide a product for the preparation of wtAAV particles and pseudotyped AAV particles, which does not have the above discussed drawbacks.

This technical problem is solved by providing the embodiments characterized in the claims. It has been found surprisingly that it was possible to solve the technical problem by using an AAV vector packaging plasmid containing the complete helper functions for packaging the vector DNA into AAV capsids, e.g. AAV-1, 2, 3, 4, 5 or 6, AAV-2 being preferred, and—for pseudotyping—AAV expression vector DNA sequences. The major advantage is here the simplification of the preparation of (pseudotyped) AAV particles which only require a single transfection of the desired cell (with the AAV vector packaging plasmid according to the invention), so that less time has to be spent and the cost is reduced. Furthermore, the problem occurring in the triple or double transfection does not apply, i.e. not all of the cells are transfected with all of the plasmids. This reduces the vector production and yields a great portion of empty capsids. It has turned out that as compared to the methods based on the double transfection, for example, the particle yield could be raised up to a factor of 10. In addition, the particles prepared with the AAV vector packaging plasmids according to the invention are free of adenovirus contamination. This simple method used as a basis the pDG helper plasmid described in German patent application 196 44 500.0-41 and having all of the AAV-2 and adenorival genes whose products are necessary for the preparation of AAV-2 vectors. To this end, the corresponding AAV expression vector DNA sequences were only cloned into the foreshortened E3 region (*E3*) of pDG. Besides, an expression cassette for the red fluorescent protein was cloned into the AAV vector packaging plasmid described in below Example 1 between the cap gene and the Ad5 gene for VA so as to obtain another advantage since the transfection efficiency of the AAV vector packaging plasmid can easily be checked visually in this way.

The subject matter of the present invention is thus an AAV vector packaging plasmid which comprises the following DNA sequences:

(a) a rep gene of AAV, a cap gene of AAV and AAV expression vector DNA sequences or (b) a complete AAV genome, as well as (c) all of the other helper virus DNA sequences which are necessary for forming AAV particles.

The rep gene and cap gene are preferably derived from the serotypes AAV-1, AAV-2, AAV-3, AAV-4, AAV-5 or AAV-6, the two genes being derivable from the same AAV serotype or from different serotypes.

The expression “helper virus DNA sequences” used herein relates to all of the DNA sequences of a helper virus which are necessary for forming AAV particles. Such DNA sequences are preferably derived from herpes viruses or adenoviruses, most preferably from adenovirus 5. The sequences may comprise the entire viral genome or fragments thereof. Suitable helper virus DNA sequences as a starting material for the preparation of the AAV vectors according to the invention are described in German patent application 196 44 500.0-41, for example, and they comprise e.g. also the DNA sequences disclosed in this patent application of the plasmid pTG 9585 which as a helper virus DNA sequence comprises the complete adenovirus 5 sequence with the exception of the E1 region. The AAV vector packaging plasmid according to the invention can also contain helper virus DNA sequences which differ from those in pTG 9585 in that they have a deletion in the structural gene L1 of Ad5 sequence, in particular in the region of nucleotides 16614-18669. Sequences for the rep and cap genes are described in the literature (rep genes and cap genes of AAV-1, AAV-3, AAV-4, AAV-5 or AAV-6 (AAV-1, Xiao et al., J. Virol. 73 (1999), 3994-4003; AAV-3, Muramatsu et al., Virol. 221 (1996), 208-217; AAV-4, Chiorini et al., J. Virol. (1997), 6823-6833; AAV-5, Bantel-Schaal et al., J. Virol. 73 (1999), 939-947; Chiorini et al., J. Virol. 73 (1999), 1303-1319; AAV-6, Rutledge et al., J. Virol. 72 (1998), 309-319).

The expression “complete AAV genome” used herein relates to the entire AAV genes necessary for the preparation of an infectious AAV virus and comprises the AAV rep and cap genes and at least one terminal repetition sequence and the control elements necessary for gene expression, e.g. promoters, splice sites, polyadenylation signals. These control elements can be exchanged by other control elements for gene expression.

The expression “AAV vector packaging plasmid” used herein does not only relate to those with the original genes listed in (a) to (c) but also to AAV vector packaging plasmids having modified genes, which include deletions or insertions of nucleotides, for example, but still encode proteins having the desired biological function. By means of common methods, a person skilled in the art can determine whether a modified gene still codes for a product having the desired biological function. The person skilled in the art also knows sources for the individual genes which are characteristic of the AAV vector packaging plasmid according to the invention. General methods known in this field can be used for the construction of AAV vector packaging plasmids which contain the above DNA sequences and, where appropriate, further sequences. These methods comprise e.g. in vitro recombination techniques, synthetic methods and in vivo recombination methods, as described in Sambrook et al., Molecular Cloning: A Laboratory Manual, 2^{nd} edition, Cold Spring Harbor Laboratory Press, Cold Spring Harbor N.Y. (1989), for example. The AAV vector packaging plasmids according to the invention can be prepared e.g. by homologous recombination in bacteria, as described by Chartier et al., J. Virol., 70 (1996), 4805-4810. For example, the pDG plasmid described in German patent application 196 44 500.0-41 can be used as the basis for an AAV vector packaging plasmid according to the invention. In this plasmid, the AAV expression vector sequences are inserted in the *E3* region, for example. Suitable AAV expression vector DNA sequences are known to the person skilled in the art (Samulski et al., J. Virol. 63 (1989), 3822-3828, Zolotukhin et al., J. Virol. 70 (1996), 4646-4653). They are preferably DNA sequences comprising at least the following DNA sequences: (a) the 5′ITR and 3′ITR of AAV-2; (b) a constitutive or inducible promoter active in mammals, and (c) a polyadenylation signal. Here, the terms “5′ITR” and “3′ITR” comprise all of the “5′ITR” and “3′ITR” sequences, respectively, which allow the vector to be replicated and packaged into viral particles and integrated into the host genome. The expression “a constitutive or induciable promoter active in mammals” used herein comprises all of the promoters permitting in mammals the transcription of the desired DNA sequence, above all those resulting in a strong expression, preferably heterologous promoters. Suitable promoters are known to the person skilled in the art and comprise e.g. the constitutive promoters CMV and cytokeratin K14 promoters or the inducible promoters MMTV (Mouse Mammary Tumor Virus), metallothionein and tetracycline-controllable promoter systems (Tet-on/-off). The AAV expression vector DNA sequences of the AAV vector packaging plasmid can also contain the desired gene to be expressed in the mammalian cells, whose expression is desirable for a gene therapy, for example. The person skilled in the art can determine by means of routine experiments a suitable site within the DNA sequence of the AAV vector for an insertion of the AAV expression vector DNA sequences, attention having to be paid when choosing the insertion site that both the functions of the AAV vector (e.g. as to packaging) and those of the AAV expression vector DNA sequences (e.g. as to the excision and expression of the foreign DNA) are not impaired considerably. The AAV expression vector DNA sequences are preferably inserted in a foreshortened E3 region (*E3*).

The helper virus DNA sequences are preferably derived from herpes virus or adenovirus, with adenovirus 5 (Ad5) being preferred.

In a particularly preferred embodiment, the AAV vector packaging plasmid according to the invention contains as helper virus DNA sequences the Ad5 genes E2A, E4 and VA, which may be derived from the pDG plasmid described in German patent application 196 44 500.0-41, for example, and which are controlled by the respective original promoter or by heterologous promoters.

In addition, the AAV vector packaging plasmid may contain a gene coding for a detectable phenotypic marker so as to prove the successful introduction of the AAV vector packaging plasmid into the target cell. In an even more preferred embodiment, the AAV vector packaging plasmid according to the invention thus contains additionally an expression cassette for the expression of a marker protein, preferably a fluorescent protein. In this connection, the term “expression cassette” refers to a combination of a gene coding e.g. for a fluorescent gene and a suitable promoter which controls this gene and a polyadenylation signal. This readily proves a transfection of the desired target cell. Examples of suitable genes coding for fluorescent proteins are rfp-(red), gfp-(green), cfp-(cyan) and yfp-(yellow) gene, rfp-(red) (Dsred-cDNA; Clontech) being preferred. Examples of suitable promoters are RSV (rous sarcoma virus) promoter, CMV (cytomegalovirus) promoter and HSV (herpes simplex virus) tk promoters, the RSV promoter being preferred. This expression cassette is inserted in the AAV vector packaging plasmid at a suitable site which can easily be determined by the person skilled in the art, preferably between the 3′ end of the cap gene and the beginning of the adenoviral VA gene, e.g. in the ClaI cleavage site. This ClaI cleavage site is present in pDG.

In another particularly preferred embodiment, the present invention relates to an AAV vector packaging plasmid, the AAV expression vector DNA sequences containing an HPV16-L1-coding DNA sequence under the control of a CMV promoter. Such an AAV vector packaging plasmid referred to as pDS2-Lh1 was deposited under DSM 14406 with DSMZ [German-type collection of microorganisms and cell cultures], Braunschweig, Germany, in accordance with the provisions of the Budapest Treaty of Jul. 17, 2001.

The subject matter of the present invention also relates to wtAAV particles, preferably wtAAV-2 particles, whose genome comprises a complete AAV genome and all of the other helper virus DNA sequences necessary for forming AAV particles and (pseudotyped) AAV particles whose capsid coat is encoded by an AAV vector packaging plasmid according to the invention and which contains AAV expression vector DNA sequences.

AAV particles according to the invention can be obtained by suitable methods, e.g. by single transfection of mammalian cells, e.g. 293 cell or 911 cells, with an AAV vector packaging plasmid according to the invention. The transfection techniques are e.g. electroporation, lipofection and preferably calcium phosphate precipitation. The achievable titer is usually between 10⁷ and 10⁹ infectious viruses/ml.

A gene therapy can be carried out with AAV particles prepared with an AAV vector packaging plasmid according to the invention, the cells being transduced by incubation with the viral particles. The cells may be present in an organism, the cells to be infected being reachable by needle injection, jet injection or particle gun. On the other hand, the cells to be transduced can also be isolated from an organism, be infected outside the organism and then be returned to the organism again. Such cells are referred to as autologous cells. Moreover, as to the organism it is also possible to use allogenic cells for the transduction. In this connection, it is favorable for these cells to belong to an HLA type corresponding to the organism. The person skilled in the art knows methods of providing cells with a certain HLA type. The wtAAV particles according to the invention, preferably wtAAV-2 particles, are also useful for the adjuvant application to chemotherapy, i.e. to the tumor therapy.

Therefore, the subject matter of the present invention also relates to a medicament which contains an AAV vector packaging plasmid and/or an AAV particle according to the invention. Here, the medicament may additionally contain a pharmaceutically acceptable carrier. Suitable carriers and the formulation of such medicaments are known to the person skilled in the art. Suitable carriers comprise e.g. phosphate-buffered saline solutions, water, emulsions, e.g. oil/water emulsions, wetting agents, sterile solutions, etc. The kind of carrier depends on how to administer the AAV vector packaging plasmid and/or AAV particle according to the invention. A suitable dosage is determined by the attending physician and depends on various factors, e.g. the patient's age, sex and weight, the severity of the disease, the kind of administration, etc. It has turned out that by means of inventive AAV vector packaging plasmids and/or particles it is possible to obtain high transduction rates with the most different cells, e.g. primary cells of the cornea epithelium or muscle cells.

### SHORT DESCRIPTION OF THE FIGURES

FIG. 1

Physical Map of the AAV Vector Packaging Plasmid pDS2-L1h XYZ (Based on pDG)

The pDS2-L1h plasmid is shown which comprises the following genes: the rep and cap genes of AAV-2, the adenovirus 5 genes also essential for an AAV-2 vector production (E2A, E4 and VA), and the gene for the red fluorescent protein (rfp). The expression of the AAV-2 rep gene is controlled by the heterologous MMTV (mouse mammary tumor virus) promoter, the expression of the AAV-2 cap gene and/or the adenoviral genes is controlled by the authentic viral promoters of AAV-2 and adenovirus 5 (not shown). The rfp gene is expressed under the control of the RSV (rous sarcoma virus) promoter. Furthermore, pDS2-L1h contains AAV-2 vector sequences consisting of two AAV-2 ITR regions (inverted terminal repeats) which flank the human-adapted gene for the capsid protein L1 of the human papilloma virus type 16. The hL1 gene is expressed under the control of the CMV (cytomegalovirus) promoter.

FIG. 2

Fluorescent Labeling of Transfected Cells

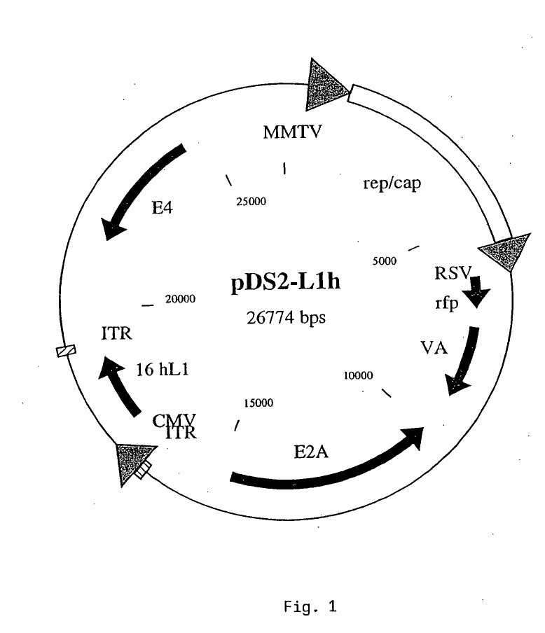

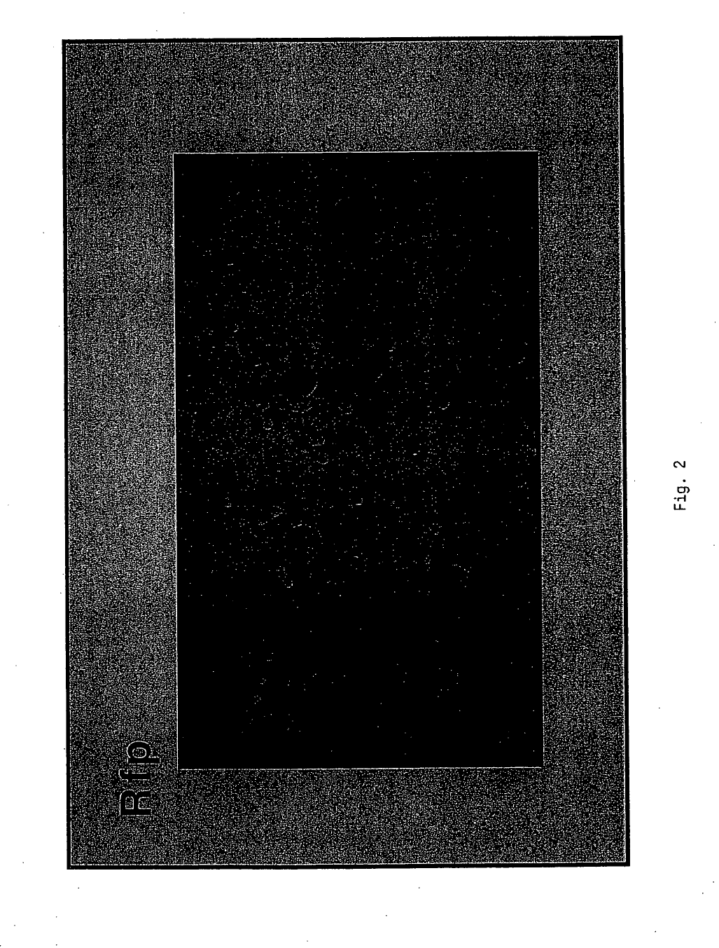

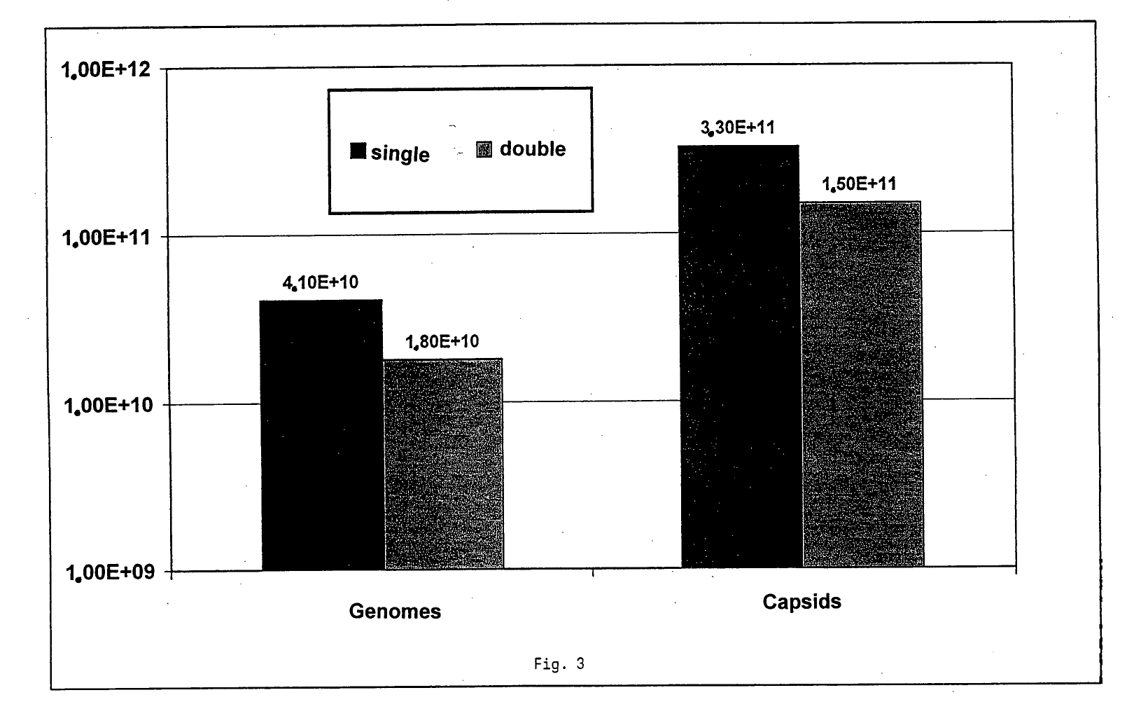

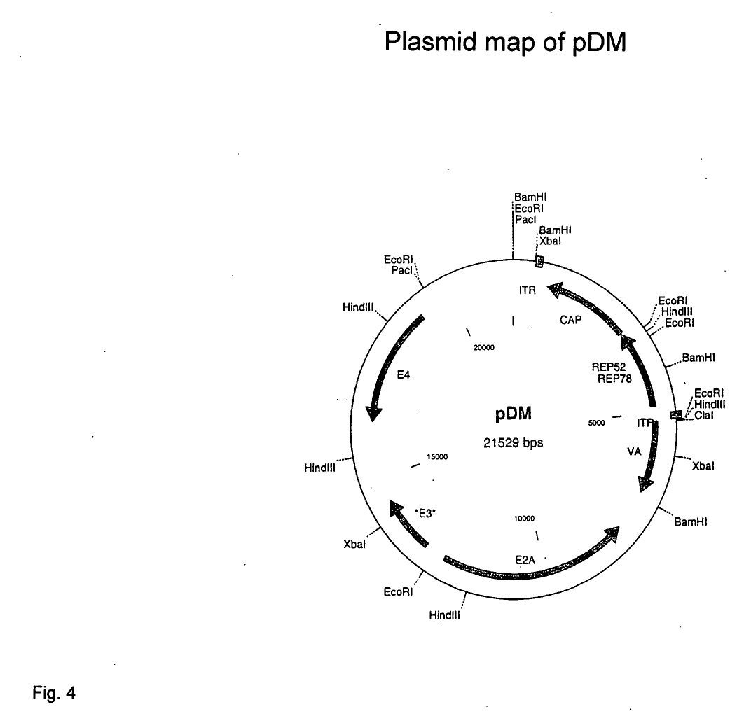

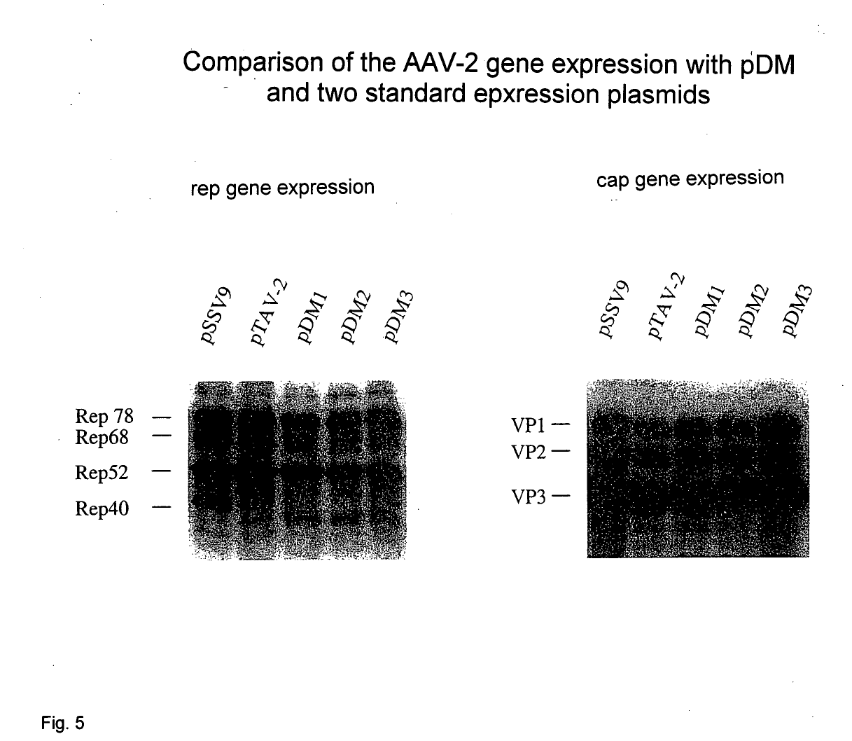

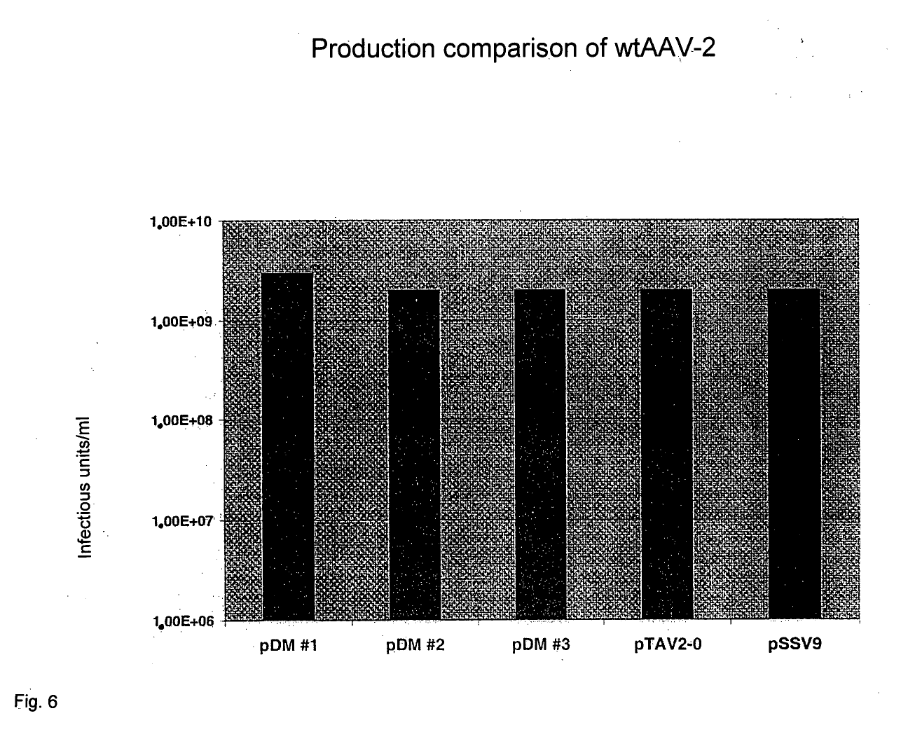

293T cells are shown which were transfected with the pDS2-L1h plasmid described in FIG. 1 (6 μg DNA, 4×10⁵ cells) and photographed under fluorescent light (at a wavelength of 558 nm for excitation of fluorescence) 48 hours after the transfection.

FIG. 3

Comparison of the Vector Production After Double Transfection or Single Transfection with an AAV Vector Packaging Plasmid According to the Invention.

The results of titration of the recombinant AAV-2 particles are shown which were obtained from the two alternative approaches, i.e. a single or double transfection. The AAV-2 vectors were quantified according to two parameters: On the one hand, the number of genome-containing (full) particles was determined (“genomes”) and, on the other hand, the entire number of viral particles (“capsids”) was determined, i.e. the sum of all full and empty particles. The titration was carried out by means of methods as described (Grimm et al., Hum. Gene Ther. 10, (1998), 2745-2760; Grimm et al., Gene Ther. 6: (1999), 1322-1330). It can be seen that the production of the AAV-2 vectors by means of single transfection was about 2.2 times more efficient.

FIG. 4

Plasmid Map of pDM

The pDM plasmid comprises the following genes: the rep and cap genes of AAV-2 flanked by both “inverted terminal repeats” (ITRs) and the adenovirus type 5 (Ad5) genes essential for an AAV-2 virus production, i.e. E2A, E4 and VA. The genes are expressed under the control of the authentic viral promoters of AAV-2 and/or Ad5 (not shown).

FIG. 5

Comparison of the AAV-2 Gene Expression with pDM and Two Standard AAV-2 Expression Plasmids

Western blot analyses with antibodies against Rep proteins (mAb 303.9) and VP proteins (mAb B1) of 293T cells are shown which were transfected with AAV-2 genome plasmids.

FIG. 6

Comparison of the wtAAV-2 Production by Means of Different AAV-2 Genome Plasmids

For a more detailed explanation see Example 6.

The invention is explained by the below examples.

### EXAMPLE 1

Production of the AAV Vctor Packaging Plasmid pDS2-L1h For the production of the AAV vector packaging plasmid pDS2-L1h XYZ, the pDG plasmid described in German patent application 196 44 500.0-41 was used as a basis. It has a total length of 21846 base pairs. Together with the MMTV promoter replacing the AAV-2 p5 promoter, the AAV genome contained in pDG has a total length of 5044 base pairs. The plasmid pDS2-L1h (see FIG. 1) was constructed by homologous in vitro recombination of an AAV-2 vector DNA (see below) with the pDF plasmid. The pDF plasmid is a derivative of pDG and additionally carries the gene for the red fluorescent protein (rfp) under the control of the RSV promoter. The vector DNA consisting of the AAV-2 inverted terminal repeats (ITR), the CMV promoter, the L1 gene of the human papilloma virus 16, and a poly(A) region (derived from the gene of the bovine growth hormone), was isolated from the pCMV-L1h plasmid (Leder et al., J. Virol. being printed). To this end, three fragments were obtained by restriction of pCMV-L1h; fragment A resulted from the restriction with PStI and NotI, fragment B was obtained by restriction with NotI and HindIII, and fragment C was obtained by restriction with Hind III and PstI. Fragment A was then inserted in the pBluescript plasmid (Stratagene) which had previously been restricted using PstI and NotI as well. Fragments B and C were jointly cloned into the pSL1180 plasmid (Pharmacia) linearized using NotI and NsiI. Thereafter, the original fragments of the L1h vector (A, B and C) could again be isolated by restriction with ClaI and NotI (fragment A from pBluescript) or NotI and NheI (fragments B and C from pSL1180). The resulting two fragments (A′ and BC′) were then cloned jointly into the pBSΔE3UFΔ plasmid. This plasmid is based on the pBSΔE3 plasmid (Grimm, D. 1998, Entwicklung und Anwendung neuartiger Methoden zur Produktion, Reinigung und Titration rekombinanter Adeno-assoziierter Viren des Typ 2 [development and use of novel methods for the production, purification and titration of recombinant adeno-associated type 2 viruses], doctoral thesis, University of Heidelberg) which comprises an 8471 base pair long Bgl 1 fragment (base pair 24876-35442 based on wild-type adenovirus 5) from adenovirus d1324 (D1324 is a mutant of adenovirus 5 where by restriction with XbaI an 1878 base pair long deletion was produced within the E3 region). In pBSΔE3, an AAV-2 vector DNA was initially inserted which had been derived from the pTR-UF3 plasmid (Zolotukhin et al., J. Virol. 70, (1996), 4646-4654). To this end, a fragment was isolated from pTR-UF3 by partial restriction using PstI and cloned into the pSL1180 plasmid linearized with PstI. Then, the AAV-2 vector DNA was isolated from the resulting plasmid as NheI/SpeI fragment and inserted in the pBSΔE3 plasmid restricted by XbaI. Finally, the resulting plasmid was excised by means of ClaI and XbaI so as to clone in the above-described fragments A′ and BC′ of the L1h vector. Then, a fragment was obtained from the resulting pBSΔE3UF plasmid by restriction using PacI, which contained the L1h vector DNA and flanking sequences of the d1324 adenovirus. This fragment was inserted by transformation together with the NdeI-linearized plasmid pDF in E. coli BJ5183 (Chartier et al., J. Virol. 70, (1986), 4805-4810). The homologous recombination, which took place in the bacteria, of the two fragments resulted in the pDS2-L1h plasmid.

### EXAMPLE 2

### Fluorescent Labeling of Transfected Cells

The pDS2-L1h plasmid carries the gene for the red fluorescent protein under the control of the RSV promoter; a transfection of cells with pDS2-L1h correspondingly results in the expression of the red fluorescent protein which fluoresces reddish-orange following excitation at a suitable wavelength (558 nm). A maximum intensity of the expression of the red fluorescent protein or a maximum intensity of fluorescence is observed 1-2 days after the beginning of transfection (cf. FIG. 2). Since each of the cells struck in the transfection of pDS2-L1h expresses the red fluorescent protein and thus fluoresces following excitation, this enables a rapid and easy check of the efficiency of the plasmid transfection which is an important parameter in the production of AAV-2 vectors.

### EXAMPLE 3

### Comparison of the Vector Production After Double or Single Transfection with the AAV Vector Packaging Plasmid Produced According to Example 1

The pDS2-L1h plasmid carries all of the genes and/or components for the production of AAV-2 vectors which comprise the gene for the L1 capsid gene of human type 16 papilloma virus. A transfection of 293T cells with pDS2-L1h thus results in the production of such vectors. In concrete terms, the following approach was tested: 4×10⁶ 293T cells (in a 15 cm culture dish) were transfected with 90 μg pDS2-L1h and then incubated at 37° C. and with 5% CO2 for 48 hours. Thereafter, the cells were collected and disintegrated according to a standard protocol (Hauswirth et al., Methods Enzymol. 316, (2000), 743-761) to release the produced AAV-2 vectors. In a second approach, the vectors were produced by means of a double transfection. To this end, 293T cells were transfected under equal conditions but in this case with 70 μg pDF and 18 μg pCMV-L1h. The cells were harvested and the viruses were released as described above. Typical results of such single or double transfection approaches are shown in FIG. 3.

### EXAMPLE 4

### Production of the pDM Plasmid and Helper Virus-Free Production of wtAAV-2 Virus

The pDM plasmid was derived from the pDG plasmid (Grimm et al., Human Gene Therapy 9, 2745-2760). The AAV-2 rep and cap genes contained therein were removed by restriction using ClaI-NdeI and XbaI-NdeI. This leads to two pDG fragments which contain the pDG helper sequences without AAV-2 rep and cap genes. The complete AAV-2 genome was isolated from the pTAV2-0 plasmid (Heilbronn et al., J. Virol. 64 (1990), 3012-3018) by restriction using XbaI-ClaI and ligated with the two pDG fragments (ClaI-NdeI and XbaI-NdeI) in a 3-fragment ligation to give pDM (FIG. 4). The PDM plasmid was deposited with DSMZ under number DSM 14927 on Apr. 10, 2002.

An expression control following transient transfection showed that by means of the pDM plasmid the Rep and VP proteins were expressed in comparable amounts with respect to the starting plasmid pATV2-0 or another reference plasmid (pSSV9, identical with psub 201; Samulski et al., J. Virol. 61 (1987), 3096-3101) (FIG. 5). Likewise, the wtAAV-2 virus production by means of the pDM plasmid is as efficient as the production by means of the plasmids pTAV2-0 or pSSV9 which additionally require an infection with Ad5 (FIG. 6). The advantage of the pDM plasmid for the production of wtAAV-2 viruses is that with equal efficiency wtAAV-2 viruses can be produced without helper viruses and thus without helper virus contamination.

### EXAMPLE 5

### Comparison of the AAV-2 Gene Expression with pDM and Two Standard AAV-2 Expression Plasmids

293T cells were cultured under standard conditions in Petri dishes (10 cm in diameter) and transfected at about 60% confluence with 12 μg DNA each according to the method by Chen and Okayama (1988, Bio Techniques 6, 632-38). In the case of the plasmids pSSV9 (corresponds to psub 201; Samulski et al. (1987)) and pTAV2-0 (Heilbronn et al., J. Virol. 64, (1990), 3012-3018) the cells were additionally superinfected with Ad5 (MOI: 5). Following transfection with the plasmids pDM1, 2 and 3 (1-3 represent different clones of the same construct) this is not necessary since the plasmids carry the AAV-2 genome and the genes for the adenoviral helper functions (see FIG. 4). The cells were scraped off 48 hours after the transfection, sedimented (300×g for 5 minutes), washed once with PBS and mixed with 500 μl 2×SDS sample buffer. Then, they were lyzed by heating them to 100° C. for 5 minutes and sonicated from outside for 20 seconds with 80% power. Cell debris was sedimented at 16500×g for 2 minutes and 15 μl of the supernatants were used for the SDS polyacrylamide gel electrophoresis. Western blot analyses with antibodies against Rep proteins are shown (mAb 303.9 Wistuba et al., J. Virol. 69, 5311-5319 (1995) and VP proteins (mAb B1 9 Wistuba et al., J. Virol. 69, 5311-5319 (1995)) of 293T cells which had been transfected with AAV-2 genome plasmids.

### EXAMPLE 6

### Comparison of the Production of wtAAV-2 by Means of Different AAV-2 Genome Plasmids

The wtAAV-2 production following transfection of 293T cells was compared with conventional AAV-2 genome plasmids (pSSV9 and pTAV2-0) and superinfection with a helper virus (Ad5; MOI:5), or after transfection with the pDM plasmid without helper virus infection (with respect to the transfection and culturing of the cells see Example 3). FIG. 6 shows the results of the titrations of the AAV-2 viruses obtained with the corresponding methods. The titration was effected via an end point determination by detecting the successful infection of HeLa cells using an AAV-2 Rep antibody (mAb 76/3 Wistuba et al., J. Virol. 69, 5311-5319 (1995)). The HeLa cells were coinfected with Ad5 and AAV-2 after serial dilution of the AAV-2 virus stock solutions. As is evident the helper virus-fee production of wtAAV-2 is as efficient as the helper virus-assisted production.

1. An AAV vector packaging plasmid comprising the following DNA sequences:
(a) a rep gene of AAV, a cap gene of AAV and AAV expression vector DNA sequences or (b) a complete AAV genome; and
(c) all of the other helper virus DNA sequences necessary for forming AAV particles.

2. The AAV vector packaging plasmid according to claim 1, wherein the helper virus DNA sequences are derived from herpes virus.

3. The AAV vector packaging plasmid according to claim 1, wherein the helper virus DNA sequences are derived from adenovirus.

4. The AAV vector packaging plasmid according to claim 3, wherein the adenovirus is adenovirus 5.

5. The AAV vector packaging plasmid according to claim 4, wherein the helper virus DNA sequences are the AdS genes for E2A, E4 and VA.

6. The AAV vector packaging plasmid according to claim 5, which additionally contains an expression cassette for the expression of a fluorescent protein.

7. The AAV vector packaging plasmid according to claim 6 6, wherein the expression cassette for the expression of a fluorescent protein is localized between the 3′ end of the cap gene and the 5′ end of the AdS gene for VA.

8. The AAV vector packaging plasmid according to claim 7, wherein the fluorescent protein is the “red fluorescent” protein.

9. The AAV vector packaging plasmid according to claim 6, wherein the fluorescent protein is functionally linked with an RSV promoter.

10. The AAV vector packaging plasmid according to claim 1 wherein the AAV expression vector DNA sequences are inserted in a foreshortened E3 region.

11. The AAV vector packaging plasmid according to claim 1, wherein the AAV expression vector DNA sequences contain a DNA sequence coding for the HPV 16-L1 protein under the control of a CMV promoter.

12. A AAV-wt genome packaging plasmid whose genome contains the DNA sequences (b) and (c) according to claim 1 to generate a wtAAV particle.

13. The AAV wt genome packaging plasmid according to claim 12 wherein the wtAAV particle is wtAAV-2.

14. A AAV particle comprising a capsid coat encoded by the AAV vector packaging plasmid according to claim 1 and which contains AAV expression vector DNA sequences.

15. A pharmaceutical composition comprising an AAV particle according to claim 12 and a pharmaceutically acceptable carrier.

16. A method for gene or tumor therapy the method comprising administering to a subject in need thereof an effective amount of an AAV particle according to claim 1.

17. A method of preparing a wtAAV particle or pseudotyped AAV particle, the method comprising:
transfecting mammalian cells with an AAV vector packaging plasmid according to claim 1; culturing the cells in a medium under suitable conditions for growth; and isolating the AAV particle from the mammalian cells or the medium.

18. The AAV vector packaging plasmid according to claim 7, wherein the AAV expression vector DNA sequences contain a DNA sequence coding for the HPV 16-L1 protein under the control of a CMV promoter.

19. The AAV vector packaging plasmid according to claim 5, wherein the AAV expression vector DNA sequences contain a DNA sequence coding for the HPV 16-L1 protein under the control of a CMV promoter.

20. A pharmaceutical composition comprising an AAV particle according to claim 5 and a pharmaceutically acceptable carrier.

21. A pharmaceutical composition comprising an AAV particle according to claim 1 and a pharmaceutically acceptable carrier.

22. A method for gene or tumor therapy, the method comprising administering to a subject in need thereof an effective amount of an AAV particle according to claim 5.
